(19) United States
(12) Patent Application Publication
(10) Pub. No.: US 2021/0225471 A1
(43) Pub. Date: Jul. 22, 2021

(54) MEDICAL REPORTING ASSISTANCE APPARATUS AND MEDICAL REPORTING ASSISTANCE METHOD

(71) Applicant: CANON MEDICAL SYSTEMS CORPORATION, Otawara-shi (JP)

(72) Inventor: Shingo TAKAGI, Otawara (JP)

(73) Assignee: CANON MEDICAL SYSTEMS CORPORATION, Otawara-shi (JP)

(21) Appl. No.: 17/154,291

(22) Filed: Jan. 21, 2021

(30) Foreign Application Priority Data
Jan. 21, 2020 (JP) 2020-007437
Dec. 15, 2020 (JP) 2020-207796

(51) Int. Cl. G16H 15/00; G06F 40/279; G16H 10/60

(52) U.S. Cl. CPC G16H 15/00; G16H 10/60; G06F 40/279

(57) ABSTRACT

A medical reporting assistance apparatus according to an embodiment includes an acquirer, an associator, and a generator. The acquirer is configured to acquire medical report data. The associator is configured to associate the medical report data with a report item. The generator is configured to associate generate output data that represents results of associations established by the associator.

### CROSS-REFERENCE TO RELATED APPLICATIONS

This application is based upon and claims the benefit of priority from Japanese Patent Applications No. 2020-007437, filed on Jan. 21, 2020 and No. 2020-207796, filed on Dec. 15, 2020; the entire contents of which are incorporated herein by reference.

### FIELD An embodiment of the present invention relates to a medical reporting assistance apparatus and a medical reporting assistance method.

### BACKGROUND

When an emergency situation arises in a hospital or other facilities, a nurse or the like may orally report condition of the patient to a doctor using a communications terminal. In such a scene, to report the condition of the patient accurately, it is considered desirable that report items include, for example, Identify, Patient, Situation, Background, Circumstances, Assessment, Request, Demand, Confirm, and Check.

### BRIEF DESCRIPTION OF THE DRAWINGS

FIG. 1 is a block diagram showing a configuration of a medical reporting assistance apparatus according to a first embodiment;

FIG. 2 is a diagram for explaining a method for assessing report data;

FIG. 3 is a diagram showing an example of assessment results displayed on a communications terminal of a reporter;

FIG. 4 is a diagram showing an example of assessment results displayed on a communications terminal of a report receiver;

FIG. 5 is a flowchart of a medical reporting assistance operation;

FIG. 6 is a block diagram showing a configuration of a medical reporting assistance apparatus according to Variation 1;

FIG. 7 is a block diagram showing a configuration of a medical reporting assistance apparatus according to Variation 2;

FIG. 8 is a diagram showing an example of a report history;

FIG. 9 is a diagram showing an exemplary layout plan of a reporting site; and

FIG. 10 is a diagram showing an example of an electronic chart stored in an electronic chart system.

### DETAILED DESCRIPTION

In an emergency scene where the condition of a patient takes a sudden turn, if even one of report items is missing, a situation can occur in which the treatment of the patient is delayed. However, in order for nursing staff and the like to become able to report all items in the emergency situation, some training and getting used to are required.

An object of the present embodiment is to provide a medical reporting assistance apparatus and a medical reporting assistance method capable of assisting accurate medical reporting.

A medical reporting assistance apparatus according to an embodiment includes an acquirer, an associator, and a generator. The acquirer is configured to acquire medical report data. The associator is configured to associate the medical report data with a report item. The generator is configured to associate generate output data that represents results of associations established by the associator.

Embodiments will now be explained with reference to the accompanying drawings. The present invention is not limited to the embodiments.

First Embodiment

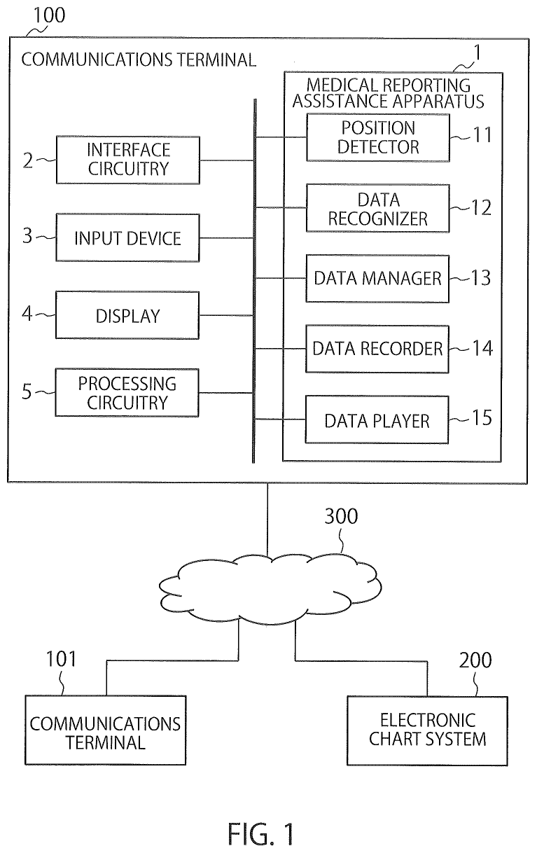

FIG. 1 is a block diagram showing a configuration of a medical reporting assistance apparatus according to a first embodiment. As shown in FIG. 1, the medical reporting assistance apparatus 1 according to the present embodiment is installed in a communications terminal 100 (first communications terminal). The communications terminal 100 is used by nurses and the like, who are reporters. The communications terminal 100 may be a smart phone or other cellular phone or a fixed-line phone installed at a predetermined place in facilities.

In addition to the medical reporting assistance apparatus 1, the communications terminal 100 according to the present embodiment includes an interface circuitry 2, an input device 3, a display 4, and a processing circuitry 5. First, these components will be described briefly.

The interface circuitry 2 is connected to the communications terminal 101 (second communications terminal) via a network 300. The communications terminal 101 is used by doctors and the like, who are report receivers. The network 300 is, for example, a LAN (Local Area Network) installed in the facilities. A configuration of the communications terminal 101 is the same as the communications terminal 100 except that the medical reporting assistance apparatus 1 is not provided. Therefore, detailed description of the configuration of the communications terminal 101 will be omitted here.

Also, the respective interface circuitry 2 of the communications terminal 100 and the communications terminal 101 are connected to an electronic chart system 200 via the network 300. Plural electronic charts are stored in the electronic chart system 200. Symptoms, treatment details, and the like of a patient are described in each electronic chart.

The input device 3 includes, for example, a touch panel displayed on the display 4, a microphone used to enter voice, and a bar code reader configured to read a bar code attached to each patient.

The display 4 is implemented, for example, by a liquid-crystal monitor. The display 4 displays various data processed by the processing circuitry 5 or medical reporting assistance apparatus 1.

The processing circuitry 5 is implemented, for example, by a processor. The processing circuitry 5 controls components of the communications terminal 100 in response to input operations performed via the input device 3.

Next, a configuration of the medical reporting assistance apparatus 1 will be described. The medical reporting assistance apparatus 1 includes a position detector 11, a data recognizer 12, a data manager 13, a data recorder 14, and a data player 15. All or part of functions of the components may be implemented either as hardware or as software.

The position detector 11 detects the current position of the communications terminal 100. The position detector 11 pinpoints the current position of the communications terminal 100 based, for example, on positions at which radio waves from transmitters (beacons) installed at various places in the facilities are received by the communications terminal 100.

Each time report data on the patient is entered via the input device 3, the data recognizer 12 assesses the report data for each of predetermined plural report items. The report data includes, for example, voice data of the reporter as well as patient identification data read from the bar code attached to the patient. According to the present embodiment, the data recognizer 12 functions as the acquirer configured to acquire medical report data including the voice data and the patient identification data, and the associator configured to associate the acquired medical report data with the report items.

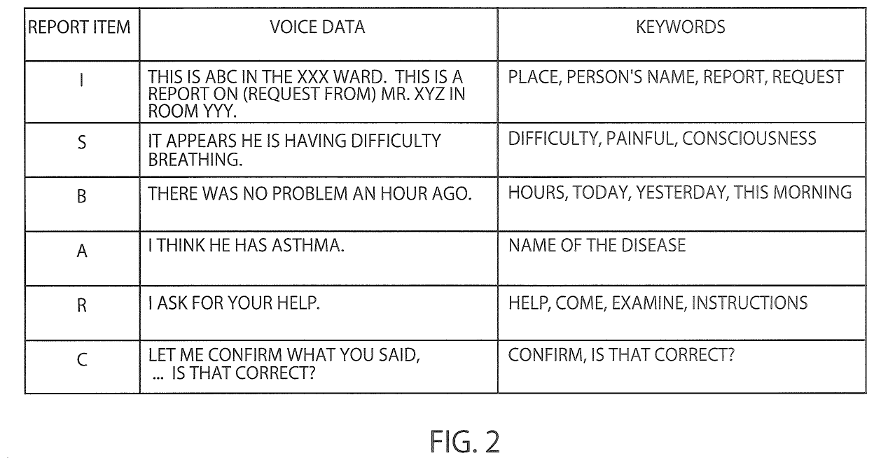

FIG. 2 is a diagram for explaining a method for assessing report data. According to the present embodiment, as shown in FIG. 2, Identify, Patient, Situation, Background, Circumstances, Assessment, Request, Demand, Confirm, and Check are determined beforehand as report items. Also, plural keywords are set beforehand for each of the report items. The data recognizer 12 converts the voice data entered via the input device 3 into text data. Next, the data recognizer 12 searches the text data for the keywords using named entity recognition. Next, the data recognizer 12 assesses the voice data based on the presence or absence of the keywords. Note that the assessment of the report data is not limited to methods that use keywords.

The data manager 13 causes the assessment results produced by the data recognizer 12 to be displayed on the respective displays 4 of the communications terminal 100 and the communications terminal 101. Also, the data manager 13 records the report data in the data manager 13 together with position data of the communications terminal 100, in other words, the position data that indicates the place where the voice data was entered. According to the present embodiment, the data manager 13 functions as the generator configured to generate output data that represents results of associations established by the data recognizer 12.

The voice data recorded in the data manager 13 can be played back by the data player 15. The voice data may contain a silent portion if the reporter was taking time in reporting or checking something. In such a case, the data player 15 may reproduce the voice data from which the silent portion is deleted. For example, the data player 15 determines a portion in which data value of the voice data remains unchanged for a predetermined period of time as being a silent portion. By deleting the silent portion, it is possible to save reproduction time. According to the present embodiment, the data player 15 functions as an output unit configured to output voice data.

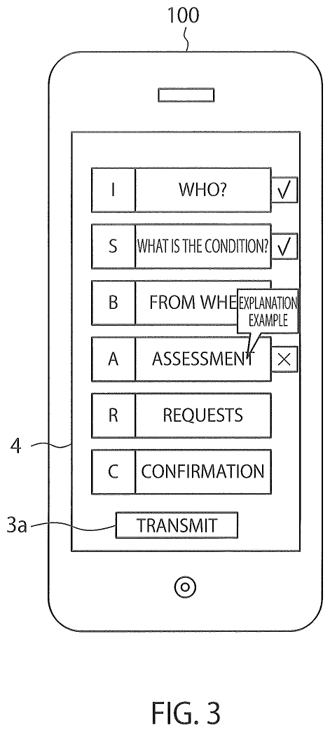

FIG. 3 is a diagram showing an example of assessment results displayed on the communications terminal 100 of a reporter. As shown in FIG. 3, on the display 4 of the communications terminal 100, an indicator that represents an assessment result produced by the data recognizer 12 is provided next to each report item. In FIG. 3, a tick (checkmark) means that the item has been reported and a cross means that the item has not been reported. That is, the data manager 13 creates image data including the report items such that each of the report items is displayed to allow for identification of whether the item has been reported and causes the image data to be displayed on the display 4 of the communications terminal 100.

Regarding item I “Who?” shown in FIG. 3, instead of using voice data, the data recognizer 12 may assess the item based on patient identification data obtained via the input device 3 by reading a bar code attached to the patient or based on the presence or absence of image data obtained by photographing the patient. Also, as shown in FIG. 3, an explanation or example of a report item may pop up on the display 4 when a button for the item is pressed, and remain on the display 4 while the button is being pressed. Furthermore, if the communications terminal 100 can be connected to a medical device for the patient to be reported, measurement values (vital values) on the medical device may be displayed on the display 4 of the communications terminal 100.

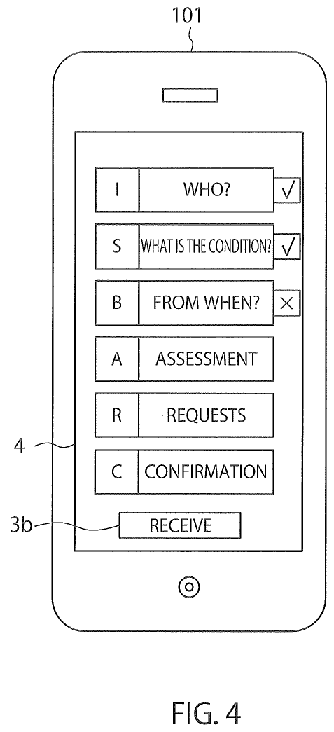

FIG. 4 is a diagram showing an example of assessment results displayed on the communications terminal 101 of a report receiver. As shown in FIG. 4, on the display 4 of the communications terminal 101, an indicator that represents an assessment result is also provided next to each report item as with the communications terminal 100. Also, when a report item button is tapped, the communications terminal 101 can display a reporting site on an in-hospital map as with the communications terminal 100. In so doing, the data manager 13 may create image data including a warning mark such that the warning mark is displayed at a reporting site having any missing item. Furthermore, vital values of the patient measured by the medical device may be transmitted from the communications terminal 100 to the communications terminal 101 and displayed on the display 4 of the communications terminal 101.

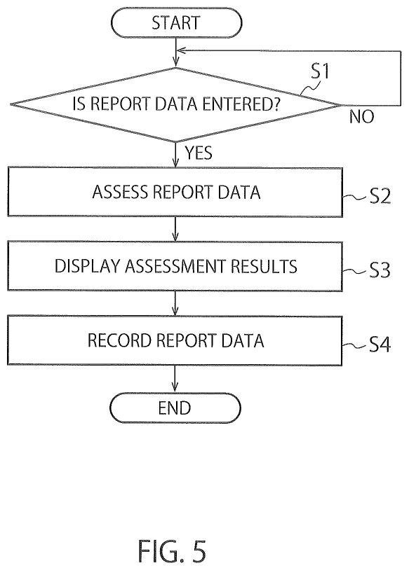

An example of a medical reporting assistance operation of the medical reporting assistance apparatus 1 will be described below with reference to FIG. 5. FIG. 5 is a flowchart of a medical reporting assistance operation.

When a nurse presses a Transmit button 3a (see FIG. 3) of the communications terminal 100, the communications terminal 100 gets ready for recording. Also, when a doctor presses a Receive button 3b of the communications terminal 101, the communications terminal 101 gets ready to receive a transmission from the communications terminal 100.

After the Transmit button 3a of the communications terminal 100 is pressed, if the nurse makes an utterance, voice data of the nurse is entered in the communications terminal 100 as part of report data (Step S1: YES). Next, the data recognizer 12 assesses the entered voice data using the method described above (step S2).

Next, the data manager 13 causes the assessment results produced by the data recognizer 12 to be displayed on the respective displays 4 of the communications terminal 100 and the communications terminal 101 (step S3). Subsequently, the data manager 13 records the voice data on the data recorder 14 together with the position data (place from which reporting was done) of the communications terminal 100 detected by the position detector 11 (step S4).

According to the present embodiment described so far, the voice data of the reporter is recognized and analyzed by the data recognizer 12 in real time, and it is determined whether content of each report item is sufficient. Subsequently, determination results are displayed on the display 4 of the communications terminal 100 of the reporter by the data manager 13. Consequently, even if any report content is missing, the reporter can learn the inadequacy of reporting at once. Thus, the medical reporting assistance apparatus 1 according to the present embodiment can assist accurate reporting complete with all the report items.

Also, according to the present embodiment, voice data and position data, which are recorded on the data recorder 14, can be played back by the data player 15. Therefore, if all the report items are not complete during actual use or training, it is possible to retrospectively check where and when risky reporting has been done. Note that when voice data is used for retrospective checks, desirably the data player 15 has capabilities to reproduce not only voiced portions, but also silent portions. This is because then it is possible to verify whether the reporting is going smoothly.

(Variation 1)

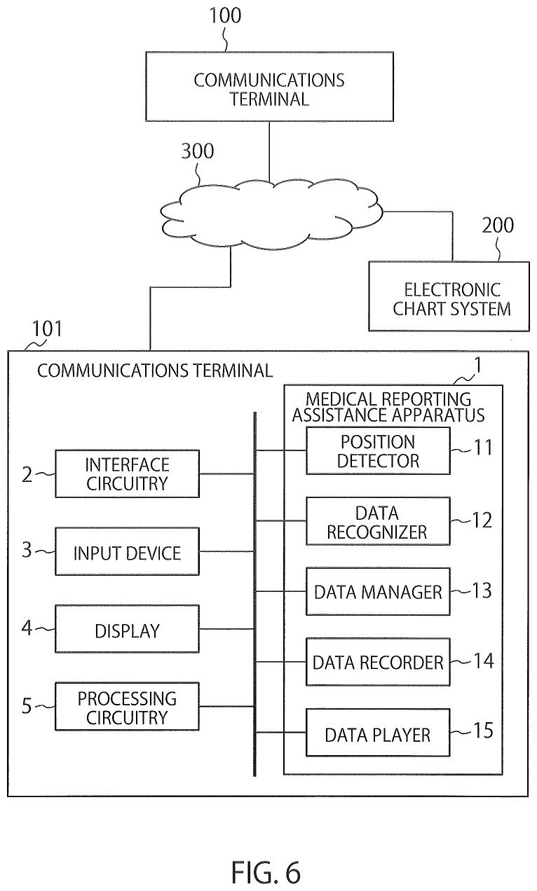

FIG. 6 is a block diagram showing a configuration of a medical reporting assistance apparatus according to Variation 1. A medical reporting assistance apparatus 1a shown in FIG. 6 is installed in the communications terminal 101 used by a doctor, who is a report receiver. Note that a configuration of the medical reporting assistance apparatus 1a is similar to that of the medical reporting assistance apparatus 1 described in the first embodiment, and thus detailed description thereof will be omitted.

According to the present variation, the communications terminal 100 transmits report data and position data to the communications terminal 101 via the network 300, where the position data indicates the current location of the communications terminal 100. In the communications terminal 101, as with the first embodiment, the data recognizer 12 assesses the voice data contained in the report data.

Next, the data manager 13 causes the assessment results produced by the data recognizer 12 to be displayed on the respective displays 4 of the communications terminal 100 and the communications terminal 101. Subsequently, the data manager 13 records the voice data contained in the report data on the data recorder 14 together with the position data (place from which reporting was done) of the communications terminal 100.

According to the present variation described above, as with the first embodiment, it is determined whether content of each report item is sufficient, and determination results are displayed on the display 4 of the communications terminal 100 of the reporter. This allows the reporter to learn any inadequacy of reporting at once and makes it possible to assist accurate reporting complete with all the report items.

Also, according to the present variation, again, voice data and position data, which are recorded on the data recorder 14, can be played back by the data player 15. Consequently, report content can be checked retrospectively.

(Variation 2)

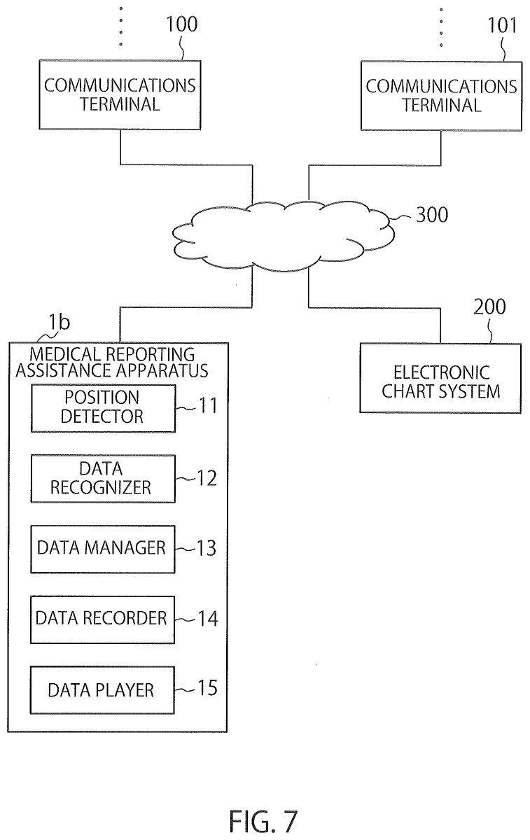

FIG. 7 is a block diagram showing a configuration of a medical reporting assistance apparatus according to Variation 2. A medical reporting assistance apparatus 1b shown in FIG. 7 functions as part of a server connected with the communications terminal 100 used by a nurse, who is a reporter, the communications terminal 101 used by a doctor, who is a report receiver, and the electronic chart system 200 via the network 300. Note that a configuration of the medical reporting assistance apparatus 1b is also similar to that of the medical reporting assistance apparatus 1 described in the first embodiment, and thus detailed description thereof will be omitted.

According to the present variation, the communications terminal 100 transmits the report data, the position data indicating the current location of the communications terminal 100, and an identification number of the communications terminal 100 to the medical reporting assistance apparatus 1b via the network 300. In the medical reporting assistance apparatus 1b, as with the first embodiment, the data recognizer 12 assesses the voice data contained in the report data.

Next, the data manager 13 transmits the assessment results produced by the data recognizer 12 simultaneously to the communications terminal 100 and the communications terminal 101 in order for the assessment results to be displayed on the respective displays 4 of the terminals. Subsequently, the data manager 13 records the voice data contained in the report data on the data recorder 14 together with the position data (place from which reporting was done) and the identification number of the communications terminal 100. In so doing, the data recorder 14 also records a report time (input time of voice data), any missing item not reported, and the like. The data recorded on the data recorder 14 can be used for retrospective checks after training or the like. Usage examples of the data will be described below.

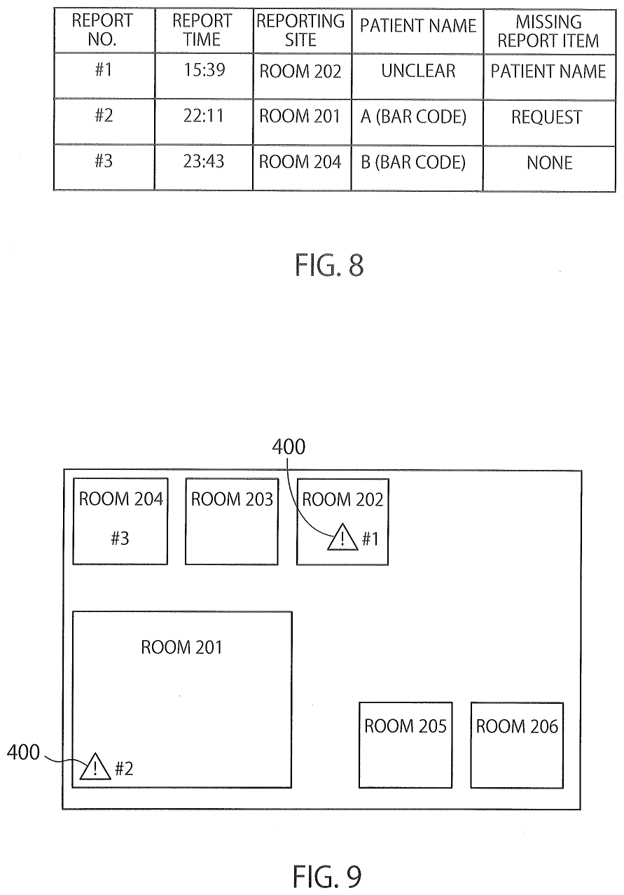

FIG. 8 is a diagram showing an example of a report history. The report history shown in FIG. 8 is displayed on the respective displays 4 of the communications terminal 100 and the communications terminal 101 by the data manager 13 based on the data recorded on the data recorder 14. According to the present variation, histories of report numbers, report times, reporting sites, patient names, and missing report items are displayed on the displays 4. This allows the nurse and doctor to see any report having any missing item at a glance.

FIG. 9 is a diagram showing an exemplary layout plan of a reporting site. The layout plan shown in FIG. 9 is also displayed on the respective displays 4 of the communications terminal 100 and the communications terminal 101 by the data manager 13 based on the data recorded on the data recorder 14. In the layout plan, a report number is displayed for each reporting site. Furthermore, if there is any missing report item, a warning mark 400 is displayed next to the report number. The warning mark 400 allows the nurse and doctor to realize a risk attached to reporting.

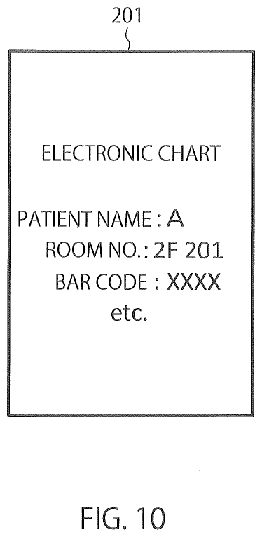

FIG. 10 is a diagram showing an example of an electronic chart stored in the electronic chart system 200. The electronic chart 201 shown in FIG. 10 is also displayed on the respective displays 4 of the communications terminal 100 and the communications terminal 101 when read out of the electronic chart system 200 by the data manager 13. In the electronic chart 201, the patient is identified by bar code data. Thus, the data manager 13 searches the report history shown in FIG. 8 for a bar code matching the bar code data of the electronic chart 201. If there is a matching bar code data, the data player 15 reproduces voice data corresponding to the bar code. This allows the nurse and doctor to check past report content about the patient.

According to the present variation described above, again, report content of the reporter is assessed in real time and assessment results are displayed immediately. This allows the reporter to learn any inadequacy of reporting at once and makes it possible to assist accurate reporting complete with all the report items.

Also, according to the present variation, again, the report data, which is recorded on the data recorder 14, can be played back by the data player 15. Consequently, report content can be checked retrospectively. In particular, according to the present variation, because the medical reporting assistance apparatus 1b is installed outside the communications terminal 100 and the communications terminal 101, even if there are plural communications terminals 100 of reporters, report data can be managed collectively.

If report data is entered from plural communications terminals 100 around the same time, the data manager 13 may, for example, numerically score assessment results on plural sets of report data and determine the order in which the sets of report data will be displayed on the communications terminal 101 of the report receiver, according to the scores. In so doing, weights may be assigned to the report items such that the scores of the assessment results will increase according to importance of the report content. Note that an identification number has been assigned to each communications terminal 100 and is transmitted to the medical reporting assistance apparatus 1b together with the report data. This makes it possible to automatically identify the reporter.

According to the embodiment and variations described above, voice data input is started at the press of the Transmit button 3a, but the method for entering voice data is not limited to this. For example, a method adopted may involve entering voice data by pressing appropriate report item buttons. In that case, because reporting can be carried out by verifying the report items one by one, missing items can be avoided.

(Variation 3)

Variation 3 will be described. According to the present variation, the medical reporting assistance apparatus 1 may be installed on the communications terminal 100 or communications terminal 101 as described in the first embodiment and Variation 1. Alternatively, the medical reporting assistance apparatus 1 may be part of the server described in Variation 2. Description will be given below, focusing on differences from the embodiment and variations described above.

According to the present variation, the data manager 13 creates image data including additional report items such that the additional report items are displayed on the display 4 of the communications terminal 100 according to report content. For example, if report data contains voice data concerning symptoms such as “have difficulty breathing” or “asthma,” the data manager 13 determines additional report items according to the symptoms.

According to the present variation, additional report items (e.g., concrete vital values) have been created beforehand on a symptom by symptom basis in a template. Therefore, the data manager 13 determines additional report items based on the template. Specifically, upon extracting information concerning symptoms such as “have difficulty breathing” or “asthma” from voice data, the data manager 13 causes additional report items to be displayed to receive more detailed information such as “frequency” and “state” associated with any item concerning the symptoms. The additional report items are managed by being associated with one another in a hierarchical structure made up, for example, of an upper layer, a middle layer, and a lower layer. For example, when information about a corresponding report item is extracted from voice data, the data manager 13 causes the additional report items associated with the report item to be displayed. This makes it possible to receive detailed information changing dynamically according to the report of the reporter. Note that the additional report items are not limited to those based on a template and do not have to be managed by being associated with one another. For example, the data manager 13 may determine additional report items based on a model learned by machine learning. In that case, when voice data concerning symptoms is entered in the learned model, the learned model outputs learned additional report items according to the symptoms.

When the additional report items are displayed on the display 4 of the communications terminal 100, the voice data of the reporter is newly entered in the communications terminal 100. On the communications terminal 100, the data recognizer 12 assesses the voice data and determines whether each of the report items has been reported.

Also, according to the present variation, the data manager 13 creates image data including plural action items such that the action items are displayed on the respective displays 4 of the communications terminal 100 and the communications terminal 101 according to report content. According to the present variation, plural action items (e.g., prescription medicines) have been created beforehand on a symptom by symptom basis in a template. Therefore, the data manager 13 determines plural action items based on the template. For example, the action items are managed by being associated with the report items. When the report items are managed by being associated with one another in a hierarchical structure as described above, the action items may be managed by being associated with one another for each of the report items. For example, when information about a corresponding report item is extracted from voice data, the data manager 13 causes plural action items associated with the report item to be displayed. Note that the action items are not limited to those based on a template. For example, the data manager 13 may determine action items based on a model learned by machine learning. When voice data concerning symptoms is entered in the learned model, the learned model outputs plural learned action items according to the symptoms.

When the plural action items described above are displayed on the respective displays 4 of the communications terminal 100 and the communications terminal 101, by operating the communications terminal 101, the doctor selects the action item considered to be the best one from among the plural action items. The selected action item is displayed on the display 4 of the communications terminal 100. The reporter gives treatment according to the displayed action item. This allows the doctor and reporter to communicate with each other appropriately and thereby take an appropriate action.

According to the present variation described above, displaying additional report items makes it possible to do more detailed medical reporting. Also, displaying action item makes it possible not only to do reporting, but also to give treatment.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A medical reporting assistance apparatus comprising:
an acquirer configured to acquire medical report data;
an associator configured to associate the medical report data with report items; and
a generator configured to generate output data that represents a result of an association established by the associator.

2. The medical reporting assistance apparatus according to claim 1, further comprising an output unit configured to output voice data contained in the medical report data.

3. The medical reporting assistance apparatus according to claim 2, wherein:
the associator associates the medical report data with the report items; and
the generator generates image data as the output data, the image data including the report items such that each of the report items is displayed to allow for identification of whether the item has been reported.

4. The medical reporting assistance apparatus according to claim 3, wherein when there is any item yet to be reported, the generator creates image data as the output data, the image data including a warning mark such that the warning mark is displayed on a layout plan of a reporting site.

5. The medical reporting assistance apparatus according to claim 3, wherein the generator creates image data as the output data, the image data including a history of the medical report data such that the history of the medical report data is displayed.

6. The medical reporting assistance apparatus according to claim 5, wherein:
the generator searches the history of the medical report data for identification data that matches identification data of a patient listed in an electronic chart; and
the output unit reproduces voice data corresponding to the identification data.

7. The medical reporting assistance apparatus according to claim 2, wherein the output unit reproduces the voice data from which a silent portion is deleted.

8. The medical reporting assistance apparatus according to claim 1, wherein the associator assesses the medical report data based on a keyword corresponding to the report item.

9. The medical reporting assistance apparatus according to claim 1, further comprising a position detector configured to detect a position of a source of the medical report data.

10. The medical reporting assistance apparatus according to claim 1, wherein the generator displays the output data on a first communications terminal used by a reporter and a second communications terminal used by a report receiver.

11. The medical reporting assistance apparatus according to claim 10, wherein the medical reporting assistance apparatus is installed on the first communications terminal.

12. The medical reporting assistance apparatus according to claim 10, wherein the medical reporting assistance apparatus is installed on the second communications terminal.

13. The medical reporting assistance apparatus according to claim 10, wherein the medical reporting assistance apparatus is connected to the first communications terminal and the second communications terminal via a network.

14. The medical reporting assistance apparatus according to claim 1, wherein the generator creates image data as the output data, the image data including an additional report item such that the additional report item is displayed according to report content of the medical report data.

15. The medical reporting assistance apparatus according to claim 14, wherein the generator creates image data as the output data, the image data including an action item such that the action item is displayed according to the report content.

16. A medical reporting assistance method comprising:
acquiring medical report data;
associating the medical report data with a report item; and
generating output data that represents a result of associating the medical report data with a report item.
